(19) United States
(12) Patent Application Publication
(10) Pub. No.: US 2024/0423777 A1
(43) Pub. Date: Dec. 26, 2024

(54) VEIN GRAFT PREPARATION PUMP

(71) Applicant: Quest Medical, Inc., Allen, TX (US)

(72) Inventors: Steve Parnis, Missouri City, TX (US); Andrew Nelson, Dallas, TX (US); Jeffrey Albertsen, McKinney, TX (US)

(21) Appl. No.: 18/749,043

(22) Filed: Jun. 20, 2024

Related U.S. Application Data
(60) Provisional application No. 63/522,566, filed on Jun. 22, 2023.

(51) Int. Cl. A61F 2/06

(52) U.S. Cl. CPC A61F 2/062

(57) ABSTRACT

The disclosed principles provide regulated pressurization and continuity/uniformity in the pressure applied through harvested veins using a unique vein graft preparation pump. Generally speaking, the apparatus incorporates one or more elastic members, each with a predetermined elastic constant or modulus of elasticity, or alternatively a collective predetermined elastic constant or collective modulus of elasticity, that is sufficient to deliver fluids from a syringe through a vessel cannula inserted into the vein section selected for a grafting procedure. Additionally, the disclosed apparatus provides only a limited amount of pressure such that no injury is caused to the vein. Pressure and flow are limited and maintained constant via use of the one or more elastic members, i.e., no pressure changes/spikes, which provides uniform pressurization at a predetermined amount safe for the harvested vein section, thereby eliminating the human error present with manual pressurization during distension of grafted veins.

### RELATED APPLICATIONS

This disclosure claims priority to U.S. Provisional Patent Application Ser. No. 63/522,566, filed Jun. 22, 2023, the entirety of which is incorporated herein by reference for all purposes.

### TECHNICAL FIELD

This disclosure relates generally to the field of vein grafting, and more particularly to a novel vein graft preparation pump for preparing veins for grafting, as well as methods of use and methods of manufacture thereof.

### BACKGROUND

Coronary artery bypass surgery, also called coronary artery bypass grafting (CABG) is a surgical procedure to treat coronary artery disease (CAD), which is, generally speaking, the buildup of plaque in the arteries of the heart. CABG and percutaneous coronary intervention (PCI) are the two methods to revascularize stenotic lesions of the cardiac arteries. PCI is a non-surgical procedure used to treat narrowing of the coronary arteries using a combination of coronary angioplasty with stenting. Conversely, a CABG procedure is a surgical procedure performed to bypass narrowing areas of the heart's arteries by using arteries or veins harvested from other parts of the body. After performing such a bypass, CABG restores adequate blood supply to the heart, thereby slowing the progression of CAD and increasing life expectancy for the patient.

A common CABG procedure involves a saphenous vein graft (SVG) for the bypassing of narrowed areas of the arteries. The great saphenous vein (or long saphenous vein) is a large, subcutaneous, superficial vein of the leg. It is the longest vein in the body, running along the length of the lower limb for returning blood from the foot, leg and thigh to the deep femoral vein. However, long-term patency (i.e., remaining sufficiently open) of SVGs has reported vein graft failure (VGF) is as high as 45% at 18 months after surgery. It is believed that such high, long-term failure rate is because preparation of SVGs before grafting leads to injury of the vein prior to grafting, which may promote VGF.

Some structural changes of the tunica intima (i.e., the innermost layer) to prevent thickening (i.e., intimal hyperplasia) and vein wall remodeling are necessary for vein graft adaptation to the arterial environment. During and after the harvesting, veins go through a period of ischemia (restriction in blood flow/supply) and reperfusion (tissue damage caused when blood supply returns to tissue after a period of lack of oxygen) after engraftment, which causes damage to endothelial cells (cells that line the interior surface of the vein) and smooth muscle cells (the cells present in the layer surrounding the tunica intima).

Veins are naturally adapted to an environment of low pressure and low blood flow; however, in a graft, veins are transferred and integrated into the arterial circulation, where they are exposed to high pressure and flow. This exposure to arterial pressure and flow causes increased shear stress and wall tension, which further damages the endothelial layer and smooth muscle cells. Over time, such continued damage results in luminal loss that makes the graft more susceptible to atherosclerosis. Progressive atherosclerosis is the primary cause of late vein graft failure.

Conventional preparation of saphenous veins for an SVG typically involves the manual distension (i.e., stretching) of the veins using fluid pressure. For example, U.S. Pat. No. 3,958,557 provides a device and method for preparing a blood vessel, such as the saphenous vein, for use as a coronary bypass graft. FIG. 1 illustrates an isometric view of a cannula 10 constructed in accordance with the principles disclosed in the U.S. Pat. No. 3,958,557. This conventional cannula 10 is comprised of a medical grade polyethylene having the flexibility and softness required to prevent trauma to the intima of the vein in which it is used. The cannula 10 has a hub 14 at the proximal end thereof which is cylindrical and terminates in a planar surface 16. The hub 14 is connected to a body 12 that is diametrically enlarged to form a shoulder 18 in a plane parallel to the surface 16. The hub 14 is sized to accommodate coupling with surgical tubing (not shown), which is press-fit on the hub 14 and abuts shoulder 18. The body 12 is frustoconical in configuration, the forwardly tapering ramp surface 20 merging with stylet 22, which forms the distal end of the cannula 10. The stylet 22 has a very gradual taper from the body 12 to the distal end 24. The external dimension of the stylet 22 is selected to be essentially the normal internal diameter of a suitable vein segment for use in coronary bypass. The stylet 22 includes a peripheral flange 26 spaced slightly behind the distal end 24. The peripheral flange 26 has a forwardly tapering ramp surface 28 that facilitates insertion of the stylet 22 into a suitable vein segment 30. The flange 26 terminates in a shoulder 32 which permits pressure-tight ligation, as described below.

FIG. 2 illustrates a side, partial cross-section view of the conventional cannula 10 taught in U.S. Pat. No. 3,958,557. The stylet 22 has an annular bore 34 that opens at the distal end 24 and is also in open communication with hollow 38 in the hub 14. The hollow 38 is coaxial with the bore 34 and is provided with a Luer taper so as to form a female coupling. Thus, the cannula 10 is press-coupled to an irrigation instrument such as a syringe 40. The selected portion of the vein 30 is then severed and the stylet 22 inserted within the lumen of the resected vein 30 until the vein 30 is pressed tightly upon the ramp surface 20. Thereafter, temporary ligation is effected by tying suture 42 tightly around the vein 30 near the flange 26. Ligation will have the effect of creating a fluid seal between the vein segment 30 and the cannula 10.

Thereafter, a syringe 40 is used for irrigating purposes to determine whether occlusions or clots remain in the vein segment 30 and, if so, to accommodate flushing of the vein 30. A distant portion of the vein segment 30 can be clamped and manual hydrostatic pressure communicated to the vein through the cannula 10 with the syringe 40. This applied pressure is used to detect leaks in the vein segment 30, particularly at the severed tributary sites. This pressure also serves to determine the distensibility index of the vein 30. If the vein 30 distends too easily under pressure, it is not suitable for the coronary artery bypass. Assuming the vein 30 has been proven under this manual pressurization, the vein 30 is used for the coronary artery bypass.

However, data has shown that acute damage to the vein graft intima can result from extensive distension of such veins during repair of the severed tributaries of the vein graft, but that this does not necessarily occur at pressure equivalent to normal arterial pressure. However, manual pressure distension does not limit or otherwise regulate the pressure of fluid applied and does not circulate the fluid during preparation. Consequently, intimal damage, although unknown to the clinician at the time, is often caused to the grafted vein when prepared using such manual procedures due to high pressurization as a result of unregulated manual pressurization techniques found in U.S. Pat. No. 3,958,557 as well as other conventional approaches. Accordingly, what is needed in the art is a device and related method for limiting the pressure of the vein graft during preparation for a vein graft to be used in a coronary artery bypass procedure, that does not suffer from the deficiencies found with conventional vein preparation approaches. The disclosed principles provide such a unique device, methods of use, and methods of manufacture thereof.

### SUMMARY

The disclosed principles prevent over pressurization of veins harvested for vein grafts by controlling the amount and the consistency of pressure applied through the harvested veins from a continuous flow of fluid through the vein graft. This combination of regulated pressurization and continuity/uniformity in the pressure applied through harvested veins is provided using a unique vein graft preparation pump constructed in accordance with the disclosed principles. Generally speaking, the apparatus incorporates one or more elastic members, each with a predetermined elastic constant or modulus of elasticity, or alternatively a collective predetermined elastic constant or collective modulus of elasticity, that is sufficient to deliver fluids from a syringe through a vessel cannula inserted into the vein section selected for the grafting procedure. Additionally, the disclosed apparatus provides only a limited amount of pressure such that no injury is caused to the vein. Pressure and flow are limited and maintained constant via use of the one or more elastic members, i.e., no pressure changes/spikes, which provides uniform pressurization at a predetermined amount safe for the harvested vein section, thereby eliminating the human error present with manual pressurization during distension of grafted veins.

Additional embodiments and advantages and variation thereof are also encompassed within the scope of the disclosed principles, and some such exemplary embodiments discussed in further detail herein.

### BRIEF DESCRIPTION OF THE DRAWINGS

The novel features believed characteristic of the disclosure are set forth in the appended claims. The disclosure itself, however, as well as a preferred mode of use, further objectives, and advantages thereof, will be best understood by reference to the following detailed description of illustrative embodiments when read in conjunction with the accompanying drawing, in which:

FIG. 1 illustrates an isometric view of a cannula constructed in accordance with the prior art, and used in the irrigation of a vein harvested for a grafting procedure;

FIG. 2 illustrates a side, partial cross-section view of the conventional cannula illustrated in FIG. 1;

FIG. 3 illustrates a front isometric view of one embodiment of a vein graft preparation pump designed and constructed in accordance with the disclosed principles;

FIG. 4 illustrates a side view of a partial assembly of the embodiment of a vein graft preparation pump illustrated in FIG. 3;

FIG. 5 illustrates an isometric view of the drive system of the embodiment of a vein graft preparation pump illustrated in FIGS. 3 and 4;

FIG. 6A illustrates a side view of one embodiment of a cap of a vein graft preparation pump designed and constructed in accordance with the disclosed principles;

FIG. 6B illustrates an isometric view of the embodiment of a cap illustrated in FIG. 6A;

FIG. 7 illustrates an isometric view of another partial assembly of another embodiment of a vein graft preparation pump designed and constructed in accordance with the disclosed principles;

FIG. 8 illustrates an exploded view of another embodiment of a vein graft preparation pump designed and constructed in accordance with the disclosed principles;

FIG. 9 illustrates one embodiment of a plunger depressor designed and constructed in accordance with the disclosed principles;

FIG. 10 illustrates another embodiment of a plunger depressor designed and constructed in accordance with the disclosed principles;

FIG. 11 illustrates another embodiment of a plunger depressor designed and constructed in accordance with the disclosed principles; and

FIG. 12 illustrates another embodiment of a plunger depressor designed and constructed in accordance with the disclosed principles.

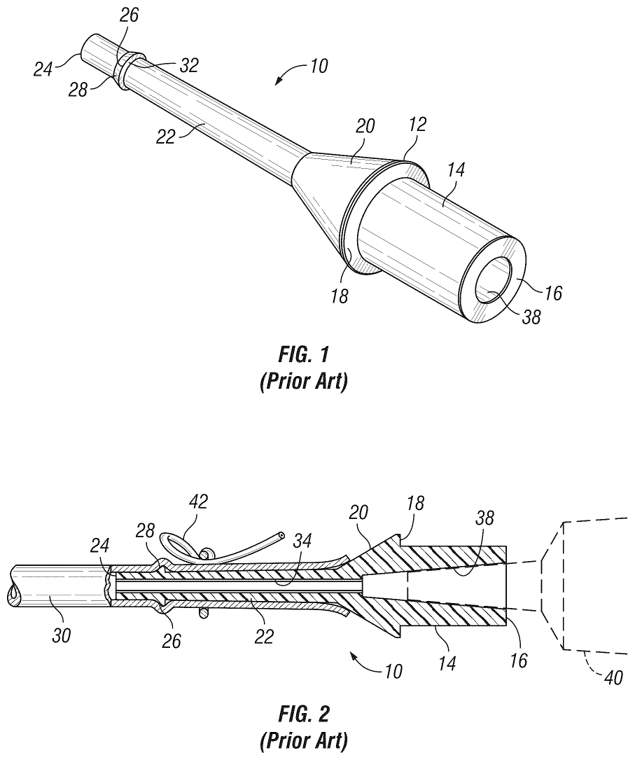

### DETAILED DESCRIPTION

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. Although multiple embodiments are shown and discussed in great detail, it will be apparent to those skilled in the relevant art that some features that are not relevant to the present invention may not be shown for the sake of clarity.

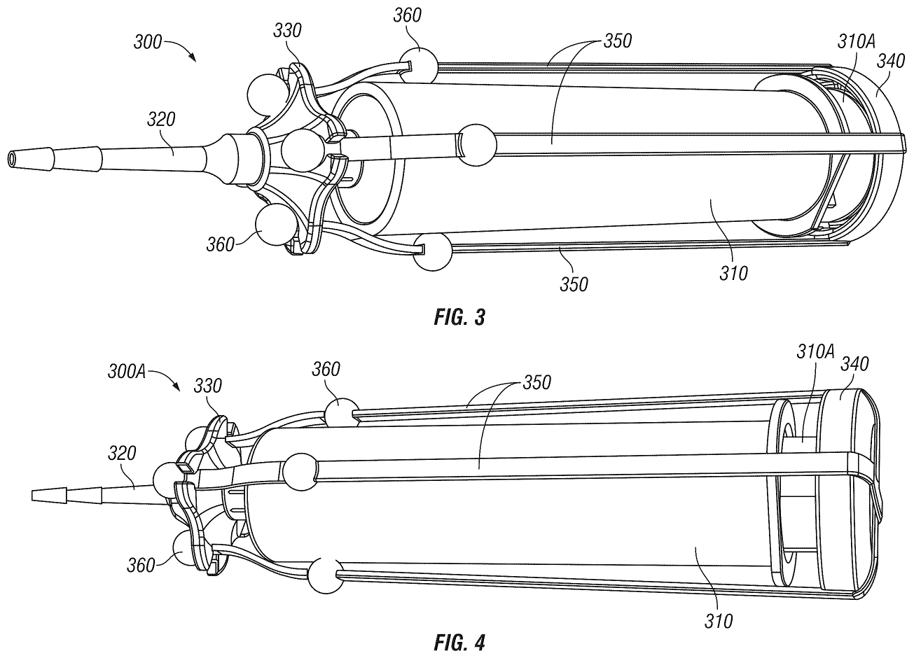

Looking now at FIG. 3, illustrated is a front isometric view of one embodiment of a vein graft preparation pump 300 designed and constructed in accordance with the disclosed principles. As mentioned above, the vein graft preparation pump 300 provides a combination of regulated pressurization and consistency in the pressure applied via a fluid pushed through harvested veins that is used in bypass procedures. This uniform pressurization at a predetermined amount safe for the harvested vein section (typically a range of about 2-4 psi, and in particular 2.7 psi) eliminates the human error that too often causes over pressurization due to manual pressurization during irrigation/distension of grafted veins.

The components of the illustrated embodiment of the vein graft preparation pump 300 include a syringe 310 used to irrigate a harvested vein section with fluid (e.g., saline) during the distension process. In exemplary embodiments, the syringe 310 is a 50 ml syringe; however, any size syringe may be employed with a preparation pump 300 as disclosed herein. At the forward end of the vein graft preparation pump 300 is a cannula 320, which is removably attached to a cap 330. The cannula 320 may be any type of cannula employable for use with a grafted vein, which is typically sutured onto the cannula 320 prior to the irrigation procedure. The cannula 320 may be removably attachable to the cap 330 via threads, press fit, or in some embodiments may simply be permanently affixed to the cap 330, or even integrally formed with the cap 330.

The vein graft preparation pump 300 also includes a plunger depressor 340 configured to engage the distal end of a plunger 310A of the syringe 310 in order to depress the plunger 310A during use of the pump 300. As the plunger 310A of the syringe 310 is depressed by the compressive force provided by a drive system of the illustrated pump 300 onto the plunger depressor 340, fluid is forced from within the syringe 310 and out through the cannula 320.

The drive system driving the plunger depressor 340 is comprised of a plurality of elastic bands 350. The elastic bands 350 include a predetermined elasticity selected to be sufficient to deliver fluids from the syringe 310 through the vessel cannula 320 inserted into the vein section selected for the grafting procedure. Elasticity is the ability of a body to resist a distorting influence or force, and to return to its original size and shape when that influence or force is removed. The elasticity of a material is quantified by its elastic modulus (or modulus of elasticity) that measures the amount of stress needed to achieve a unit of strain for that material. A higher modulus indicates that the material is harder to deform, and the typical unit of modulus of elasticity is the pascal (Pa). In embodiments of vein preparation pumps designed and constructed in accordance with the disclosed principles, the modulus of elasticity is selected based on several factors, including the number of elastic bands 350 employed in the pump 300, the length of the elastic bands 350, the length of the syringe 310 and plunger 310A (i.e., the length the elastic bands 350 are required to stretch), the viscosity of the fluid used for the vein irrigation, and any other factors influencing the amount of pressure applied by the elastic bands to depress the syringe depressor 310A.

Included on the elastic bands 350 are at least one band retention device 360 to retain each respective band 350 to the cap 330, thus stretching each band 350 from the plunger depressor 340 to the cap 330. In the illustrated embodiment of the pump 300, band retention devices 360 are included at the distal ends of each elastic band 350 to attach each band 350 to the cap 330. More specifically, in the illustrated embodiment, the band retention devices 360 are provided as spheres or balls which are sized to seat into corresponding spots on the side of the cap 330 opposite the location of the elastic bands 350. Of course, other shapes for the band retention devices 360 may also be employed. Also in this illustrated embodiment of the pump 300, an additional set of band retention devices 360 is provided at the same position along the length of each elastic band 350. Such additional sets of band retention devices 360 may be provided to allow repositioning of the elastic bands 350 to correspondingly affect the amount of compressive pressure that is applied to the plunger 310A of the syringe 310 by the elastic bands 350. This adjusting may be by further stretching or loosening of the elastic bands 350, depending on the location of such additional sets of band retention devices 360, which is described in further detail below. Also, two more additional sets of band retention devices 360 may also be positioned on the elastic bands 350 for even further adjustment of the pressure provided to the fluid exiting the syringe 310.

The disclosed apparatus provides only a limited amount of pressure such that no injury is caused to the vein. This combination of regulated pressurization and continuity in the pressure applied through harvested veins results in a constant, safe pressure applied from the vein graft preparation pump to the harvested veins. Pressure is limited and maintained constant via use of the elastic bands 350, which provide uniform pressurization at a predetermined amount safe for the harvested vein section (i.e., typically in the range of 2-4 psi, and preferably about 2.7 psi), thereby eliminating the human error present with manual pressurization during distension of grafted veins. To do so, the elastic bands 350 are selected to provide the desired or required pressure, with a higher modulus of elasticity providing a greater amount of pressure, while a lower modulus of elasticity will provide a lower pressure of the fluid injected into the harvested vein for irrigation.

In some embodiments, an adjustment of the elastic bands 350 may be provided in the pump 300, such as the presence of additional band retention devices 360, can adjust the amount the elastic bands 350 are pulled/stretched up to the cap 330. Doing so would increase the pressure provided by the elastic bands 350 during use of the pump 300, if needed or desired. Similarly, such a pressure adjustment mechanism could be used to decrease the pressure provided by the elastic bands 350 by including retention devices 360 that permit lengthening of the elastic bands 350 such that they are stretched less when connecting to the cap 330. Altering the amount that the elastic bands 350 are stretched from the plunger depressor 340 to the cap 330 is advantageous in order to compensate for changes in one or more variables. In one specific embodiment, multiple sets of band retention devices 360 are included along the length of each elastic band 350, and each set of retention devices 360 may include labeling on or near them corresponding to one or more another components influencing the amount of pressure produced by the preparation pump 300. For example, different sets of band retention devices 360 may be coupled to the cap 330 based on the size of the output end of the syringe 310, the size of the inner diameter of the cannula 320, or both.

Alternatively or additionally, different sets of band retention devices 360 may be coupled to the cap 330 based on the viscosity of the irrigation fluid to be disbursed by the syringe 310. In some embodiments, a chart may be provided so that a user of a pump 300 as disclosed herein will know which set of band retention devices 360 should be coupled to the cap 330 based on their particular application. For example, such a chart could set forth that if a particular size and gauge of syringe 310 is used with saline as the fluid, along with a particular gauge of cannula 320, then the first set of band retention devices 360 should be coupled to the cap 330 in order to provide a desired amount of fluid pressure (e.g., 2.7 psi for vein irrigation with saline). However, if the same size/gauge syringe 310 and cannula 320 are used, but a fluid with a higher viscosity is used (e.g., within a given range set forth on the chart), then the second set of band retention devices 360 should be coupled to the cap 330 in order to provide a desired amount of fluid pressure for the particular application of the pump 300. Such variations and possible adjustments of the elastic bands 350 permit uses of a pump designed and constructed in accordance with the disclosed principles in a wide variety of applications beyond just vein preparation.

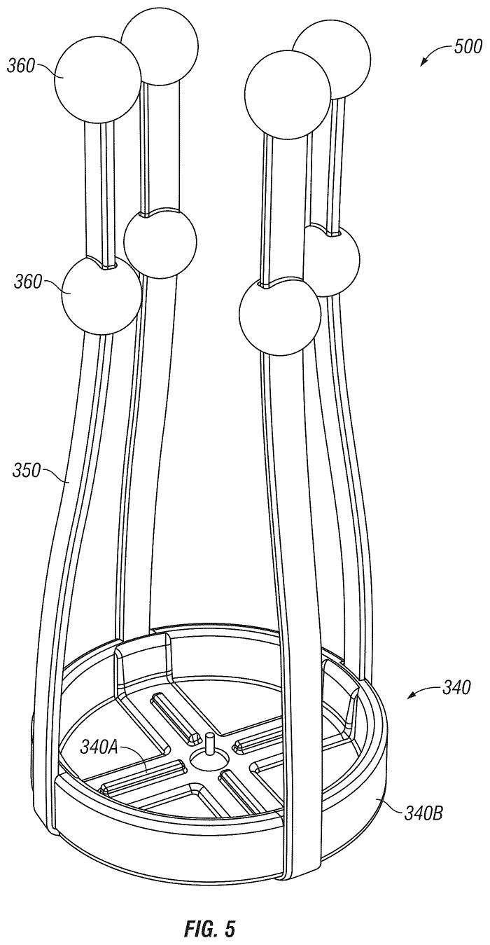

Turning now to FIG. 4, illustrated is a side view of a partial assembly 300A of the embodiment of a vein graft preparation pump 300 illustrated in FIG. 3. The view of the pump 300 illustrates the bottom side of the cap 330, as well as a backside of the plunger depressor 340. Looking at FIG. 5, illustrated is an isometric view of the drive system 500 of the vein graft preparation pump 300 illustrated in FIGS. 3 and 4. The drive system 500 is comprised of the elastic bands 350 having their distal ends fastened, either removably or permanently, to the plunger depressor 340. As discussed above, proximal ends of the elastic bands 350 are fastened (again, either removably or permanently) to the cap 330 via the retention devices 360. Doing so with a syringe 310 placed between the cap 330 and the plunger depressor 340 extends the elastic bands 350 around the syringe 310. Fastening the elastic bands 350 to the cap 330 via retention devices 360 located away from the proximal ends of the elastic bands 350, similarly extends the elastic bands 350 around the syringe 310 but with a greater amount of pressure due to an increase in the stretching of the bands 350 from the prior attachment position on the cap 330.

The interior surface of the plunger depressor 340, in this illustrated embodiment of the drive system 500, also includes raised features 340A designed to secure the distal end of a syringe plunger 310A to the plunger depressor 340. Specifically, the raised features 340A are sized and shaped to be received within such a distal end of a plunger 310A. For example, many distal ends of conventional syringe plungers typically include a uniquely shaped recess, or multiple recesses, formed inwardly on their distal ends. In many cases, this recess is a “+” shape, typically corresponding to the cross-sectional shape of the body of the syringe plunger. Providing the raised features 340A with substantially the same shape and size as the recess(es) formed in conventional syringe plungers results in the raised features 340A being positionable (e.g., press-fit) into such a recess. Also, the plunger depressor 340 may include a raised lip 340B surrounding the perimeter of the depressor 340B. Such a raised lip 340B may be sized to slip-fit around the exterior edge of the plunger 310A in order to secure the plunger 310A laterally on the plunger depressor 340. Thus, the syringe plunger 310A may be held securely onto the plunger depressor 340 with one or both of the raised features 340A and the raised lip 340B.

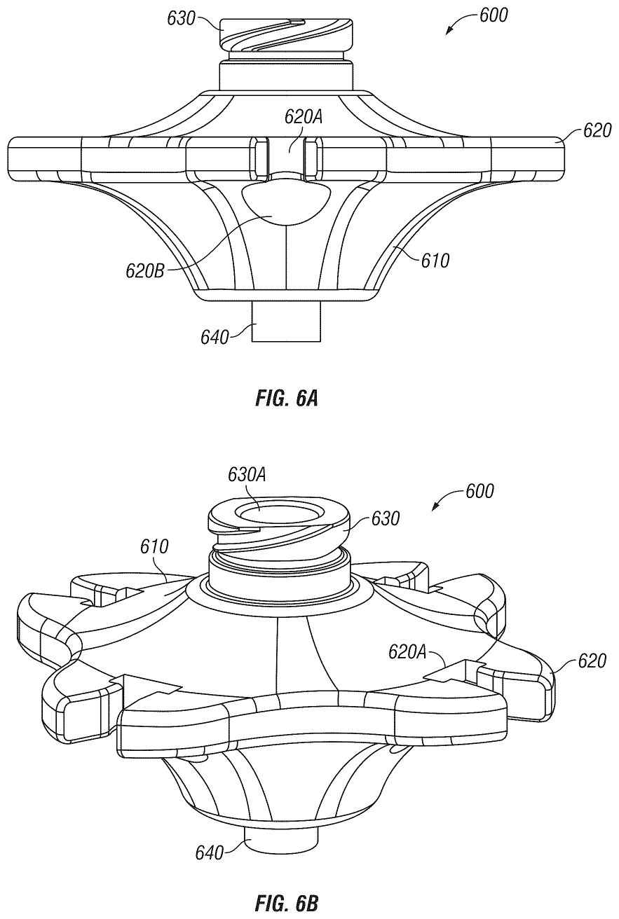

FIG. 6A illustrates a side view of one embodiment of a cap 600 of a vein graft preparation pump designed and constructed in accordance with the disclosed principles. This embodiment of a cap 600 includes a cap body 610 and flanges 620 extending from the body 610. In this embodiment, the cap 600 includes four flanges 620 corresponding to four elastic bands (not illustrated) to be connected to the cap 600. Of course, any number of flanges 620 may be provided in accordance with the number of elastic bands to be provided with a pump as disclosed herein.

At the distal ends of the flanges 620 are retention slots 620A formed therein and sized to receive and secure therein respective elastic bands. In this embodiment, the retention slots 620A are substantially formed as “T-slots” for use with elastic bands having a rectangular or flat cross-section. However, in embodiments where the cross-section of the elastic bands is a different shape, the retention slots 620A may be correspondingly shaped to receive respective bands therein. On one side of the retention slots 620A are retention seats 620B. In this embodiment, the seats 620B are semi-spherical shaped for receiving correspondingly shaped retention devices, such as devices 360 discussed above and having a spherical shape. In other embodiments, the seats 620B may have any advantageous shape corresponding to the shape of the retention devices located on the elastic bands (not illustrated).

The cap 600 also includes a proximal end 630 configured to be connected to the output tip of a syringe (not illustrated), such as the syringe 310 illustrated and discussed above. In some embodiments, the proximal end 630 includes threads that correspond to mating threads formed in the tip of the syringe onto which the cap 600 will be placed. As such, the cap 600 is removably connected to the syringe, but in other embodiments the cap may be permanently affixed to the tip of the syringe, or may even be integrally formed with the syringe tip. The cap 600 also includes a distal end 640 configured to be connected to the input end of a cannula (not illustrated), such as the cannula 320 illustrated and discussed above. In some embodiments, the distal end 640 may include threads (not illustrated) that correspond to mating threads formed in the input end of the cannula that will be placed on the cap 600. In other embodiments, the distal end 640 may be sized for a press-fit with the input end of the cannula, and other means of connection between the distal end 640 of the cap 600 and a cannula may also be provided with a pump as disclosed herein. In either such embodiment, cannula is removably connected to the cap 600, but in other embodiments the cap may be permanently affixed to the input end of a cannula, or may even be integrally formed with the cannula.

FIG. 6B illustrates an isometric view of the embodiment of a cap illustrated in FIG. 6A. From this perspective, the fluid passage 630A through the cap 600 can be seen. The fluid passage 630A passes through from the proximal end 630 of the cap 600 to and out of the distal end 640 of the cap 600. As discussed above, the inner diameter of the fluid passage 630A is sized as one of the components of a preparation pump in accordance with the disclosed principles so that the desired pressure of the fluid output from the cap and cannula is consistently maintained. In some embodiments, the proximal and distal ends 630, 640 of the cap 600, and the fluid passage 630A size, are standardized to the size of syringe that is used with the pump so that only the elasticity of the elastic bands and/or various retention devices 360 are selected or adjusted so that the pump provides the specific consistent pressure desired. Of course, in other embodiments, any such parameters or variables may be selectable to provide a desired pressure through the cannula.

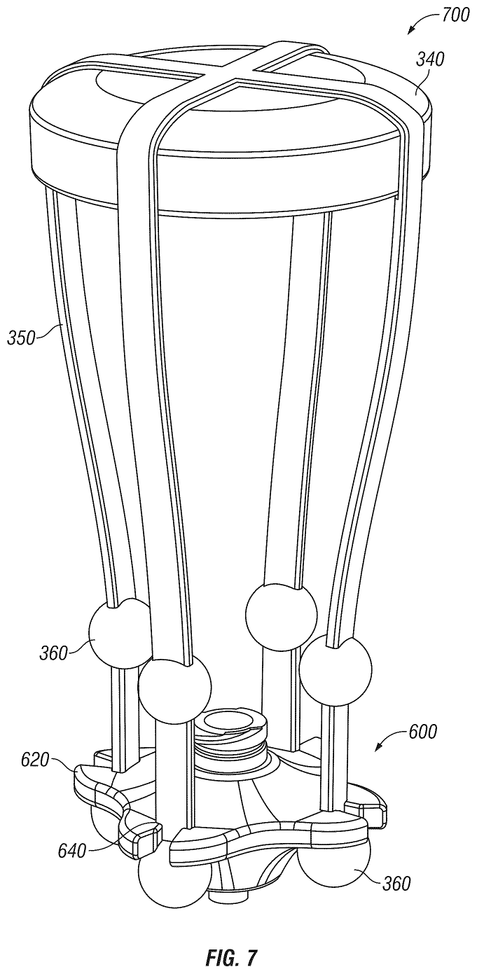

Turning now to FIG. 7, illustrated is an isometric view of another partial assembly 700 of another embodiment of a vein graft preparation pump designed and constructed in accordance with the disclosed principles. In this embodiment, the plunger depressor 340 and the elastic bands 350 surrounding and holding the plunger depressor 340 are again shown. Also, the two sets of retention devices 360 at the ends of the elastic bands 350 are also visible. Moreover, one set of retention devices 360 are shown as received within the slots 640 and seated in the retention seats 650 (not visible from this perspective) of the cap 600. As discussed in detail above, once a syringe (not illustrated) is placed within the partial assembly 700, the elastic bands are stretched taut, and when the plunger of that syringe is drawn back for use, the elastic bands are stretched even more such that the contraction of the bands causes the plunger to depress and thus the syringe to deliver the fluid therein at a consistent and maximum pressure.

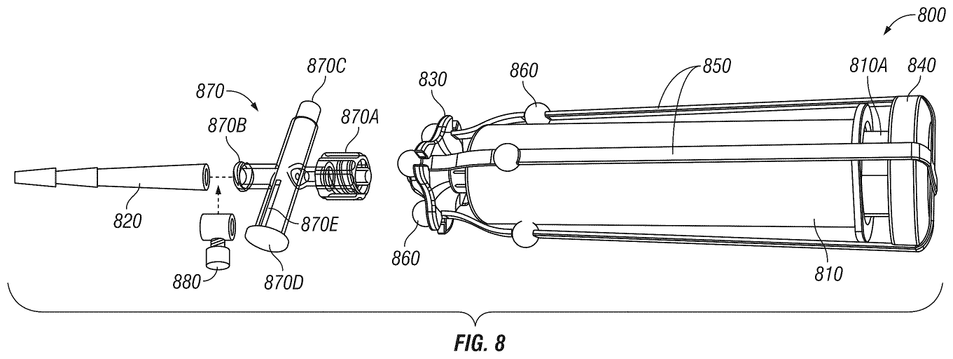

Turning to FIG. 8, illustrated is an exploded view of another embodiment of a vein graft preparation pump 800 designed and constructed in accordance with the disclosed principles. This embodiment of a vein graft preparation pump 800 again includes a syringe 810, again having a plunger 810A, for delivering fluid for irrigating an object, such as a grafted vein section. In addition, this embodiment again includes a cannula 820 and a cap 830 affixed to a delivery or output end of the syringe 810. A plunger depressor 840 is also provided, which is used to depress the plunger 810A with the compressive/pulling force provided by a plurality of elastic bands 850. A plurality of retention devices 860 are also present, again provided as two sets of devices 860. A first set of retention devices is shown retained by the cap 830 to provide a first compressive force based on the elasticity of the bands 850 (as well as other factors/variables discussed above), while the second set are provide shorter on the bands 850 to provide a greater compressive force on the syringe 810 or to provide the same compressive force as the first set of devices 860 when a shorter syringe is employed with the pump 800.

In some embodiments, a preparation pump 800 in accordance with the disclosed principles may be employed with a frame or similar structure (not illustrated) to support the pump 800 therein. For example, such a structure may be the same or similar to one provided as the “syringe holder 400” disclosed in co-pending patent application Ser. No. ______/______,______, Attorney Docket No. AQMED.0195P, filed Jun. 22, 2023, the entirety of which is incorporated herein by reference for all purposes. In such embodiments, the support structure could be configured hold the flanges of the base of the syringe at one end and the cap 830 at the opposite end. As such, the plunger depressor 840 may be permitted to move within the support structure or from outside the support structure and move towards the end of the support structure holding the syringe flanges. In other embodiments, the support structure includes a moveable component holding the plunger depressor 840, where the plunger depressor 840 can move within the support structure, such as sliding along a pair of parallel rails, to depress the plunger 810A. Of course, any type of support structure can be employed to hold the syringe as well as various components of the pump 800 so that pump 800 may simply be laid on a table or other surface, as opposed to being held, while in operation.

Also provided in this embodiment of the pump 800 is a control valve 870. The control valve 870 is provided to stop the output of fluid from the syringe 810 either prior to use of the pump 800 or while the pump 800 is being used in an irrigation procedure. While the control valve 870 can take the form of any type of valve, in this illustrated embodiment, the control valve 870 is provided as a stopcock 870. The illustrated stopcock 870 comprises an input 870A configured to be attached to the output end of the cap 830. As shown, the input 870A may include a threaded retention nut configured to thread onto mating threads on the output of the cap 830. In other embodiments, the input 870A may simply comprise threads mating with those on the output of the cap 830 such that the stopcock 870 is rotated to thread it onto the output of the cap 830. Of course, any type of connection structure may be included on the input 870A, including being sized for a press-fit onto the cap 830, as desired.

The stopcock 870 also includes an output 870B in fluid connection with the input 870A. The output 870B is configured to be attached to the input end of the cannula 820. As shown, the output 870B may comprise threads mating with those on the input of the cannula 820, where the cannula 820 (and/or the stopcock 870) may be rotated to connect the cannula 820 to the output 870B. In other embodiments, a threaded retention nut such as the one included on the input 870A may be employed on the output 870B to connect the stopcock 870 to the cannula 820. Of course, any type of connection structure may be included on the output 870B, including being sized for a press-fit onto the cannula 820, as desired.

In the depicted embodiment, the stopcock or control valve 870 includes a plug 870C with a plug aperture 120 that passes through the plug 870C from one side to the other. The plug 870C is slidably engaged within a valve plug body so that the control valve 870 can be placed in the open position or in the closed position, as shown in FIG. 8. In an embodiment in which the plug 870C is cylindrical and the valve plug body defines a cylindrical cavity sized to receive the plug 870C, control valve 870 can include an alignment interface 870E to prevent the plug 870C from rotating within the valve plug body. In this illustrative embodiment, the alignment interface 870E includes an alignment rail that is received within an alignment channel.

The plug 870C also includes a plug head 870D, which can receive a pressing force to cause the control valve 870 to transition from the closed configuration to the open configuration. In the depicted embodiment, the plug head 870D has a diameter that is larger than a diameter of the valve plug body to limit travel of the plug 870C so that fully depressing the plug head 870D causes an aperture formed in the plug 870C to align with the fluid conduit. The control valve 870 is in the open position when this aperture is aligned with the fluid conduit. In the depicted embodiment, the control valve 870 is in the open position when the plug 870C is maximally depressed. The control valve 870 will remain in the open position until a force applied to the plug 870C causes the control valve 870 to assume the closed position, i.e., when the control valve aperture is not aligned with the fluid conduit therein. Alternatively, a grasping force applied to the plug head 870D and a pulling force exerted to it can also cause the control valve 870 to assume the closed position. In the closed position, the aperture is not aligned with the fluid conduit, which prevents fluid flow past plug 870C.

As mentioned above, the stopcock or control valve 870 may be used to prevent and allow fluid to be expelled by the syringe 810 and out of the cannula 820 for an irrigation or distension procedure. Advantageously, this stopping capability of this embodiment of the pump 800 not only permits the syringe 810 to be loaded with a fluid by drawing back its plunger 810A and then preventing the compression of the elastic bands 850 from expelling that fluid until desired, but also permits a user to close the stopcock 870 to cease expelling the fluid during use of the pump 800. This locking/stopping capability also stops the fluid flow from the syringe 810 sufficiently to eliminate internal pressure to the vein if perforations in the vein are detected during irrigation/preparation. As such, any such perforations may be sutured or otherwise repaired while the pressure from the pump/syringe is eliminated by the locking/stopping mechanism 870.

Furthermore, in some embodiments of a preparation pump 800 in accordance with the disclosed principles, a fail-safe pressure release valve 880 may be included. Such a pressure release valve 880 may be used to ensure a maximum pressure (which may be determined by the type/size of harvested vein, the type/viscosity of the fluid be used, or any other factor or combination of factors) is not exceeded when employing the pump 800 to prepare the harvested vein. Such a pressure release valve 880 may take the form of valve located at the tip of the syringe or between the stopcock 870 and the cannula 820, as illustrated, which will automatically open to release pressure at a predetermined pressure amount. For example, in one embodiment, such a pressure relief valve 880 may be configured to open if the pressure exceeds about 4 psi. In a more specific embodiment, such a pressure relief valve 880 may be configured to open if the pressure exceeds about 2.7 psi. Alternatively, the valve 880 may instead be provided as a pressure regulator rather than a relief valve. In such embodiments, the pressure regulator 880 will permit flow through the vein 630 at only the desired pressure (e.g., about 2.7 psi) or at a desired pressure range (e.g., 2-4 psi). In yet other embodiments, a combination of pressure relief valve and a pressure regulator may be employed, which can be beneficial in situations where the regulator fails at the desired pressure regulation, and thus the pressure relief valve provides a safety redundancy in preventing over-pressurization of a vein during its irrigation.

Additionally or alternatively, a damper (not illustrated) may also be provided with the pump 800. An exemplary damper may employ a piston and accompanying fluid selected so that only a maximum amount of pressure can be applied with the movement of the syringe depressor during use of the pump 800. A damper may also assist in ensuring that a consistent and uniform amount of pressure is provided by the pump 800, where the elastic bands 850 alone may not provide the same amount of pressure at the beginning of their contraction than is applied at the end of their contraction. Also, such a damper could include an adjustment mechanism, which would permit a user of a pump 800 as disclosed herein to adjust the amount of pressure being applied during use, while also ensuring that amount of pressure does not exceed a maximum and is provided uniformly during use. Moreover, such a damper may be positioned in any advantageous location around the pump 800 without departing from the scope of the principles disclosed herein.

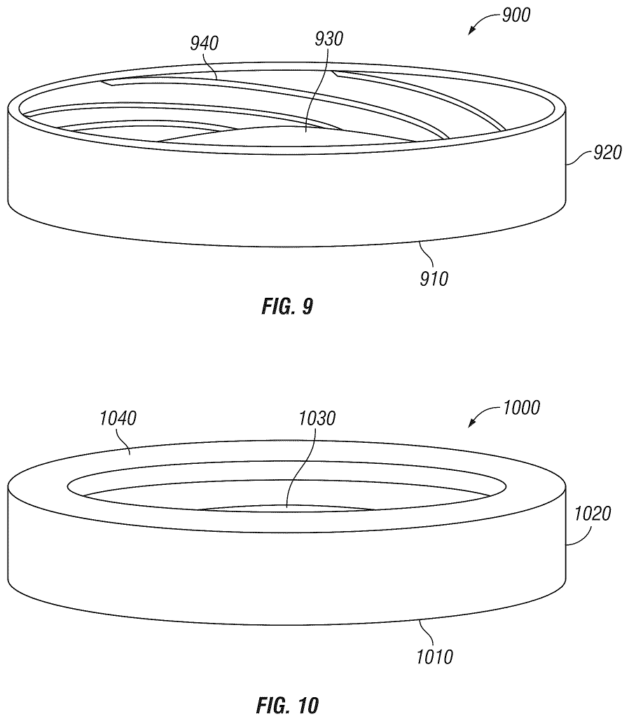

Looking now at FIG. 9, illustrated is one embodiment of a plunger depressor 900 designed and constructed in accordance with the disclosed principles. This particular embodiment of the plunger depressor 900 includes a depressor body 910, which comprises a sidewall 920 and a base 930 affixed at a bottom edge of the sidewall 920. In this embodiment, the sidewall 920 is circular in shape, but in other embodiments the sidewall 920 may take any shape. For example, the sidewall 920 may take any shape usable with a plunger base located at one end of a plunger, which could be formed with a circular, oval, or rectilinear shape.

Also in this illustrated embodiment, the plunger depressor 900 includes threads 940 formed on the interior surface of the sidewall 920. In exemplary embodiments, the sidewall 920 is circular such that a plunger base (not illustrated) may be threaded into the plunger depressor 900 until it sits against the interior of the base 930. In this manner, the plunger depressor base, and thus the plunger itself, is firmly secured within the plunger depressor 900 during use of a vein graft preparation pump in accordance with the principles disclosed herein. As such, elastic bands (not illustrated) as disclosed herein may be placed over or affixed to the plunger depressor 900 in the manner disclosed herein.

Referring now to FIG. 10, illustrated is another embodiment of a plunger depressor 1000 designed and constructed in accordance with the disclosed principles. This embodiment of a plunger depressor 1000 again includes a depressor body 1010 that also comprises a sidewall 1020 and a base 1030 affixed at a bottom edge of the sidewall 1020. As with other embodiments, the sidewall 1020 is circular in shape, as illustrated, but in other embodiments the sidewall 1020 may again take any shape. For example, the sidewall 1020 may take any shape usable with a plunger base, which as stated above could be formed having a circular, oval, or rectilinear shape.

Also in this embodiment of the plunger depressor 1000, instead of including threads formed on the interior surface of the sidewall 1020, this embodiment includes a lip or shelf 1040. Specifically, in this embodiment, the lip 1040 is formed extending inwardly from a top edge of the sidewall 1020. However, in other embodiments, the lip 1040 may extend inwardly from any position along the sidewall 1020. Functionally, the lip 1040 is sized so as to retain a plunger base between the depressor base 1030 and the interior, underside of the lip 1040. Because this results in the opening through the lip 1040 being smaller in diameter (i.e., size) than the diameter or width of the plunger base, the lip 1040 may be formed from flexible material, such as rubber, such that the plunger base may be fit within the plunger depressor 1010 and under the lip 1040. Similarly, the distance between the interior surface of the base 1010 and the bottom surface of the lip 1040 may be sized to tightly hold the plunger base so that it does not move vertically within the plunger depressor 1000. In some embodiments, the lip 1040 may be formed of such flexible material, and then affixed (e.g., via adhesive or even removably attached) to the top edge (or interior surface) of the sidewall 1020, which may be constructed of a different, perhaps more rigid, material. In other embodiments, the lip 1040 and remainder of the plunger depressor 1000 are integrally formed of a single material. Of course, any material or combination of materials may be used to form the lip 1040 and the remainder of the plunger depressor 1000.

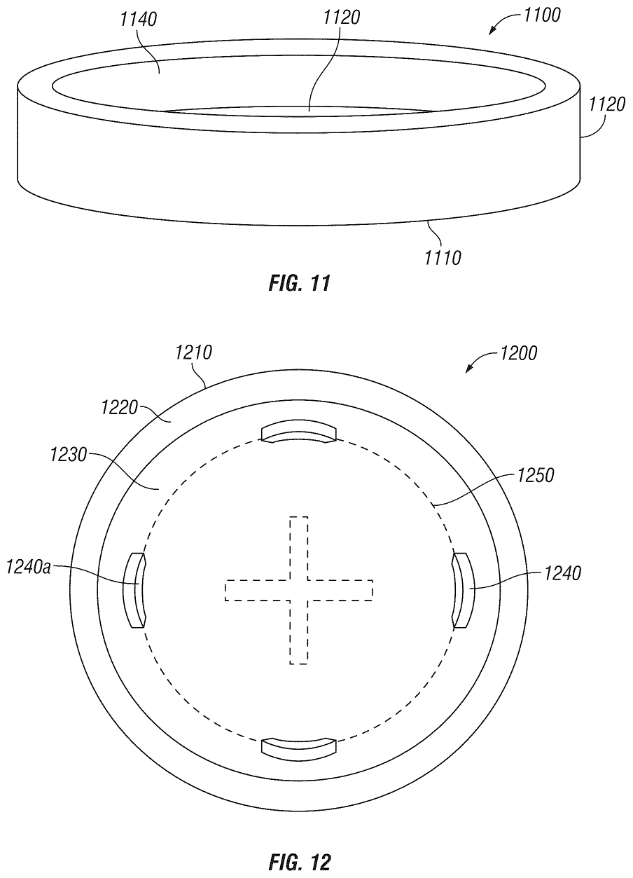

Referring now to FIG. 11, illustrated is another embodiment of a plunger depressor 1100 designed and constructed in accordance with the disclosed principles. This embodiment of a plunger depressor 1100 again includes a depressor body 1110 that also comprises a sidewall 1120 and a base 1130 affixed at a bottom edge of the sidewall 1120. As with other embodiments, the sidewall 1120 is circular in shape, as illustrated, but in other embodiments the sidewall 1120 may again take any shape usable with a plunger base.

In this embodiment of the plunger depressor 1100, instead of including threads formed on the interior surface of the sidewall 1120 or a lip formed on a top edge thereof, this embodiment includes a cavity 1140 formed into the plunger depressor 1100 down to the base 1130. Specifically, the cavity 1140 is formed downwardly from a top edge of the sidewall 1120 down to the base 1130. In some embodiments, the cavity 1140 is formed straight and thus perpendicular to the interior surface of the base 1130. In other embodiments, the cavity 1140 is formed tapered inwardly when moving from the top edge of the sidewall 1120 down to the interior surface of the base 1130.

Functionally, the interior diameter or non-circular size of the cavity 1140 is sized so as to retain a plunger base within the interior walls of the cavity 1140 and at the depressor base 1130. This results in a tight press fit of the plunger base with the plunger depressor 1100 so that the depressor 1100 does not fall off of the plunger base. As with other embodiments, the depressor 1100 may be formed from flexible material, such as rubber, such that the plunger base may be fit tightly within the cavity 1140 and against the interior of the base 1130. In some embodiments, the cavity 1140 may be formed of such flexible material, and then affixed (e.g., via adhesive or even removably attached) within the sidewall 1120 of the depressor 1100, which may be constructed of a different, perhaps more rigid, material. In other embodiments, the cavity 1140 and remainder of the plunger depressor 1100 are integrally formed of a single material. Of course, any material or combination of materials may be used to form the cavity 1140 and the remainder of the plunger depressor 1100.

In some embodiments, various sized rings (not illustrated) may be placed within the cavity 1140, where each ring (or other adapter if not circularly shaped) includes a different interior diameter or size to accommodate different sizes and/or shapes of plunger bases. In such embodiments, a user of a vein grafting preparation pump as disclosed herein may select the appropriate size of ring or adapter based on the size and/or shape of the plunger base to be employed with the pump, and insert it into the cavity 1140, e.g., also with a press fit. In other embodiments, the ring or adapter may instead be threaded into the cavity 1140, which would include corresponding threads, or snap fit therein, or using any advantageous type of fit to hold the ring or adapter within the cavity 1140. The plunger base may then be press fit into the opening provided by the selected and installed ring or adapter.

In other embodiments, the cavity 1140 may be formed with descending steps moving downwardly from the top edge of the sidewall 1120. In such embodiments, the various steps provide diameters/sizes of decreasing sizes when moving downwardly into the depressor 1100 so that different size plunger bases can be press fit into the corresponding diameter/size of step. Also in such embodiments, when a plunger base is press fit into a step diameter/size larger than the step size at the interior of the base 1130, the plunger base will seat onto the top edge of the corresponding step rather than against the interior of the base.

Looking now at FIG. 12, illustrated is another embodiment of a plunger depressor 1200 designed and constructed in accordance with the disclosed principles. This embodiment of a plunger depressor 1200 again includes a depressor body 1210 that also comprises a sidewall 1220 and a base 1230 affixed at a bottom edge of the sidewall 1220. As with other embodiments, the sidewall 1220 is circular in shape, as illustrated, but in other embodiments the sidewall 1220 may again take any shape usable with a plunger base.

In this embodiment of the plunger depressor 1200, instead of including threads formed on the interior surface of the sidewall 1220 or a lip formed on a top edge thereof, this embodiment includes arms 1240 extending upwardly from an interior of the base 1230. These arms include lips or tabs 1240a extending inwardly from each of the arms 1240. These arms 1240 and tabs 1240a work together to receive and retain a plunger base 1250 within the interiors of the arms 1240 and against the depressor base 1230. More specifically, the arms 1240 may be formed of a partially flexible material, such as plastic, so that they flex outwardly as the plunger base 1250 is pushed against the tips 1240a and towards the base 1230. The resilience of the arms 1240 causes them to move back to their original position once the plunger base 1250 moves past and under the tips 1240a. This again results in a tight press fit of the plunger base 1250 within the plunger depressor 1200 so that the depressor 1200 does not fall off of the plunger base 1250.

As with other embodiments, the depressor 1200 and the arms 1240 may be formed from the same semi-rigid material, such as plastic, such that the plunger base 1250 may be fit tightly within the arms 1240 and seat against the interior of the depressor base 1230. In some embodiments, the arms 1240 may be formed of such flexible material, and then affixed to the base 1230 of the depressor 1200, which may be constructed of a different material. In other embodiments, the tips 1240a may be constructed of a more flexible material than the arms 1240, and the two joined together via adhesive or other means. Of course, any material or combination of materials may be used to form the arms 1240, the tips 1240a, and/or the remainder of the plunger depressor 1200.

While this disclosure has been particularly shown and described with reference to preferred embodiments, it will be understood by those skilled in the pertinent field art that various changes in form and detail may be made therein without departing from the spirit and scope of the invention. The inventors expect skilled artisans to employ such variations as appropriate, and the inventors intend the invention to be practiced otherwise than as specifically described herein. Accordingly, this disclosure includes all modifications and equivalents of the subject matter recited in the claims appended hereto, as permitted by applicable law. Moreover, any combination of the above-described elements in all possible variations thereof is encompassed by the disclosure unless otherwise indicated herein or otherwise clearly contradicted by context.

Also, while various embodiments in accordance with the principles disclosed herein have been described above, it should be understood that they have been presented by way of example only, and not limitation. Thus, the breadth and scope of this disclosure should not be limited by any of the above-described exemplary embodiments, but should be defined only in accordance with any claims and their equivalents issuing from this disclosure. Furthermore, the above advantages and features are provided in described embodiments, but shall not limit the application of such issued claims to processes and structures accomplishing any or all of the above advantages.

The uses of the terms “a” and “an” and “the” and similar references in the context of describing the invention(s) (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein.

Additionally, the section headings herein are provided for consistency with the suggestions under 37 C.F.R. 1.77 or otherwise to provide organizational cues. These headings shall not limit or characterize the invention(s) set out in any claims that may issue from this disclosure. Specifically, and by way of example, although the headings refer to a “Technical Field,” the claims should not be limited by the language chosen under this heading to describe the so-called field. Further, a description of a technology as background information is not to be construed as an admission that certain technology is prior art to any embodiment(s) in this disclosure. Neither is the “Summary” to be considered as a characterization of the embodiment(s) set forth in issued claims. Furthermore, any reference in this disclosure to “invention” in the singular should not be used to argue that there is only a single point of novelty in this disclosure. Multiple embodiments may be set forth according to the limitations of the multiple claims issuing from this disclosure, and such claims accordingly define the embodiment(s), and their equivalents, that are protected thereby. In all instances, the scope of such claims shall be considered on their own merits in light of this disclosure, but should not be constrained by the headings set forth herein.

Moreover, the Abstract is provided to comply with 37 C.F.R. § 1.72 (b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the claims reflect, inventive subject matter lies in less than all features of a single disclosed embodiment. Thus, the following claims are hereby incorporated into the Detailed Description, with each claim standing on its own as a separate embodiment.

Any and all publications, patents, and patent applications cited in this disclosure are herein incorporated by reference as if each were specifically and individually indicated to be incorporated by reference and set forth in its entirety herein.

What is claimed is:

1. A vein graft preparation pump, comprising:
a cap, comprising:
a fluid passage extending from an interior face to an exterior face,
a proximal end extending from the interior face and configured to engage an output end of a syringe, and
a plurality of recess seats formed on the exterior face,
a plunger depressor having an interior surface configured to engage a plunger base of the syringe; and
a drive system driving the plunger depressor towards the interior surface of the cap, the drive system comprising:
a plurality of elastic bands coupled to the plunger depressor,
at least one band retention device included on each of the elastic bands and configured to be secured in a respective recess seat to retain each respective band to the cap, and
wherein each of the plurality of elastic bands has a modulus of elasticity sufficient to drive the plunger depressor towards the cap so as to drive fluid from the syringe at a substantially constant pressure.

2. The vein graft preparation pump of claim 1, further comprising a plurality of retention slots corresponding to and extending from respective recess seats through the cap to the interior face, wherein a portion of respective elastic bands adjacent to corresponding band retention devices passes through a respective retention slot to the interior surface of the cap.

3. The vein graft preparation pump of claim 1, wherein the band retention devices are spheres sized to seat into correspondingly shaped recess seats.

4. The vein graft preparation pump of claim 3, further comprising a plurality of second band retention devices provided at equivalent positions along a length of each respective elastic band, wherein said second band retention devices permit adjusting a length of the elastic bands extending between the cap and the plunger depressor, said adjusting affecting the amount of compressive force applied to the plunger base of the syringe by the elastic bands.

5. The vein graft preparation pump of claim 1, wherein the cap comprises four flanges corresponding to four recess seats, and wherein the drive system comprises four corresponding elastic bands.

6. The vein graft preparation pump of claim 1, wherein the retention slots comprise a T-shape and the elastic bands comprise a rectangular cross-section.

7. The vein graft preparation pump of claim 1, wherein the proximal end comprises threads that correspond to mating threads on the output end of the syringe.

8. The vein graft preparation pump of claim 1, further comprising a cannula coupled to the exterior face and fluid passage of the cap.

9. The vein graft preparation pump of claim 8, further comprising a control valve positioned between the exterior face of the cap and the cannula to stop and start output of fluid from the syringe.

10. The vein graft preparation pump of claim 9, wherein the control valve is a stopcock.

11. The vein graft preparation pump of claim 1, wherein an interior surface of the plunger depressor comprises raised features configured to secure a distal end of the plunger base to the plunger depressor.

12. The vein graft preparation pump of claim 12, wherein the raised features comprise a + shape.

13. The vein graft preparation pump of claim 1, wherein an interior of the plunger depressor comprises a raised lip surrounding the perimeter of the plunger depressor, said raised lip sized to slip-fit around an exterior edge of the plunger base to secure the plunger base laterally.

14. The vein graft preparation pump of claim 1, wherein an interior of the plunger depressor comprises a sidewall having threads formed on an interior surface of the sidewall and configured to threadably engage the plunger base to secure the plunger base laterally.

15. The vein graft preparation pump of claim 1, wherein an interior of the plunger depressor comprises a sidewall and a lip extending inwardly from a top edge of the sidewall, wherein the lip is sized to retain the plunger base between a base of the plunger depressor and an underside of the lip to secure the plunger base laterally.

16. The vein graft preparation pump of claim 1, wherein an interior of the plunger depressor comprises a sidewall having an inner diameter sized to frictionally engage the plunger base to secure the plunger base laterally.

17. The vein graft preparation pump of claim 1, wherein an interior of the plunger depressor comprises a base having arms extending upwardly from the base, wherein the arms each comprise a tab extending inwardly, the plurality of tabs configured to receive and retain the plunger base within the interiors of the arms and against the base to secure the plunger base laterally.

18. The vein graft preparation pump of claim 1, The vein graft preparation pump of claim 1, wherein the modulus of elasticity of the elastic bands is selected such that saline is output from the syringe in the range of 2-4 psi.

19. A vein graft preparation pump, comprising:
a cap, comprising:
a fluid passage extending from an interior face to an exterior face,
a proximal end extending from the interior face and configured to engage an output end of a syringe, and
a plurality of recess seats formed on the exterior face,
a plunger depressor having an interior surface configured to engage a plunger base of the syringe;
a cannula coupled to the exterior face and fluid passage of the cap;
a control valve positioned between the exterior face of the cap and the cannula to stop and start output of fluid from the syringe; and
a drive system driving the plunger depressor towards the interior surface of the cap, the drive system comprising:
a plurality of elastic bands coupled to the plunger depressor,
at least one band retention device included on each of the elastic bands and configured to be secured in a respective recess seat to retain each respective band to the cap, and
wherein each of the plurality of elastic bands has a modulus of elasticity sufficient to drive the plunger depressor towards the cap so as to drive fluid from the syringe at a substantially constant pressure.

20. The vein graft preparation pump of claim 19, further comprising a plurality of second band retention devices provided at equivalent positions along a length of each respective elastic band, wherein said second band retention devices permit adjusting a length of the elastic bands extending between the cap and the plunger depressor, said adjusting affecting the amount of compressive force applied to the plunger base of the syringe by the elastic bands.
